(19) United States
(12) Patent Application Publication
(10) Pub. No.: US 2024/0410871 A1
(43) Pub. Date: Dec. 12, 2024

(54) METHOD FOR DETERMINING TOTAL SOLUBLE SALT CONTENT IN SALINE SOIL, AND METHOD FOR EVALUATING SOIL TYPE AND SALINIZATION DEGREE OF SALINE SOIL

(71) Applicant: Gansu Province Transportation Planning, Survey & Design Institute Co.Ltd., Lanzhou City (CN)

(72) Inventors: Xiaomin LI, Lanzhou City (CN); Jingzhuo ZHAO, Lanzhou City (CN); Xuejuan WANG, Lanzhou City (CN); Dingbang WEI, Lanzhou City (CN); Jinfeng FENG, Lanzhou City (CN); Hailin WANG, Lanzhou City (CN); Jiang FAN, Lanzhou City (CN); Xu WU, Lanzhou City (CN); Mingang SHAO, Lanzhou City (CN)

(21) Appl. No.: 18/481,488

(22) Filed: Oct. 5, 2023

(30) Foreign Application Priority Data
Jun. 9, 2023 (CN) 2023106781776

(51) Int. Cl. G01N 33/24

(52) U.S. Cl. CPC G01N 33/24

(57) ABSTRACT

Provided are a method for determining a total soluble salt (TSSC) in a saline soil, and a method for evaluating a soil type and a salinization degree of a saline soil. Based on a principle for measuring a refractive index, as well as a relationship that refractive index is directly proportional to a solution concentration, the TSSC is rapidly determined through measured refractive index and a linear relationship between the refractive index of a saline soil leachate and the TSSC. The TSSC is quickly determined by using a portable refractometer; and contents of chloride ions and sulfate ions are quickly determined by using a portable chloride ion electrode-sensing analyzer and a portable barium ion electrode-sensing analyzer, respectively. In this way, rapid determination of TSSC and evaluation of salinization degree in the saline soil are realized at a construction site.

### CROSS REFERENCE TO RELATED APPLICATION

This patent application claims the benefit and priority of Chinese Patent Application No. 2023106781776 filed with the China National Intellectual Property Administration on Jun. 9, 2023, the disclosure of which is incorporated by reference herein in its entirety as part of the present application.

### TECHNICAL FIELD

The present disclosure relates to the technical field of saline soil test, in particular to a method for determining a total soluble salt content (TSSC) in a saline soil, and a method for evaluating a soil type and a salinization degree of a saline soil.

### BACKGROUND

Saline soils are widely distributed all over the world. Subsidence of highway roadbed caused by salt expansion of the saline soil is one of the main disease forms of highway in saline soil areas. The salinization degree determines the roadbed construction and treatment measures of highway in saline soil areas.

In the prior art, the soil type and salinization degree of the saline soil are mostly evaluated based on the contents of chloride ions and sulfate ions in soluble salts and a total soluble salt content (TSSC).

The methods for determining a TSSC of a saline soil specified in the highway industry specification include mass methods and conductivity methods. In the mass methods, the saline soil is mixed with water, and an obtained clear filtrate is subjected to rotatory evaporation to dryness and weighed to calculate the TSSC. This method takes 5 h to 6 h, and has low practicability on the construction site.

The contents of the chloride ions and the sulfate ions are generally determined by silver nitrate titration or ethylenediaminetetraacetic acid (EDTA) indirect coordination titration. These methods show cumbersome testing processes and long testing time.

### SUMMARY

In view of this, an object of the present disclosure is to provide a method for determining a total soluble salt content (TSSC) in a saline soil, and a method for evaluating a soil type and a salinization degree of a saline soil. In the present disclosure, the methods make it possible to quickly obtain the TSSC of the saline soil, and quickly determine the soil type and the salinization degree of the saline soil.

To achieve the above object, the present disclosure provides the following technical solutions:

The present disclosure provides a method for determining a TSSC in a saline soil, including the following steps:
mixing a saline soil to be tested with water, and conducting solid-liquid separation to obtain a saline soil leachate; and
measuring a refractive index of the saline soil leachate by using a calibrated refractometer, and obtaining the TSSC of the saline soil to be tested according to a predetermined standard curve and the refractive index of the saline soil leachate measured;
wherein the TSSC is expressed by a mass percentage content; and
the standard curve is a linear relationship curve between the TSSC and the refractive index.

In some embodiments, the water is distilled water free of carbon dioxide.

In some embodiments, a mass ratio of the saline soil to be tested to the water is in a range of 1:(1-5).

In some embodiments, the standard curve is obtained by a process including the following steps:
providing a series of saline soil leachates with a concentration gradient having known soluble salt contents as standard samples to be tested; and
measuring refractive indexes of the standard samples to be tested, and plotting the standard curve with the refractive index as an abscissa and the soluble salt content as an ordinate.

In some embodiments, the refractometer is a digital-display probe-type refractometer.

The present disclosure further provides a method for evaluating a soil type and a salinization degree of a saline soil, including the following steps:
determining a TSSC of the saline soil to be tested by the method as described in above technical solutions;
adding a barium chloride solution to a saline soil leachate, and determining a sulfate ion content in the saline soil to be tested by using a barium ion electrode-sensing analyzer;
determining a chloride ion content in the saline soil to be tested by using a chloride ion electrode-sensing analyzer;
calculating a ratio k of the chloride ion content to the sulfate ion content;
evaluating the soil type of the saline soil to be tested according to the ratio k, wherein under the condition that the ratio k is greater than 2, the soil type of the saline soil to be tested is chlorine saline soil; under the condition that the k ratio is not less than 1 and not more than 2, the soil type of the saline soil to be tested is sub-chlorine saline soil; under the condition that the k ratio is larger than or equal to 0.3 and less than 1, the soil type of the saline soil to be tested is sulfite saline soil; and under the condition that the ratio k is less than 0.3, the soil type of the saline soil to be tested is sulfate saline soil; and
evaluating the salinization degree of the saline soil to be tested with the TSSC and the soil type of the saline soil to be tested according to the “Code for Highway Engineering Geological Investigation”.

In some embodiments, determining a sulfate ion content in the saline soil to be tested includes the following steps:
mixing the saline soil to be tested with water, and conducting solid-liquid separation to obtain the saline soil leachate, where a mass ratio of the saline soil to be tested to the water is denoted as q;
adding 5 to 10 drops of a hydrochloric acid solution into the saline soil leachate with a volume of V₁, heating to boiling, adding a barium chloride solution with a volume of V₂ and a molarity of C₁ thereto, reheating a resulting mixture to boiling, and allowing to stand and cooling, to obtain a pretreated solution; and
measuring a molarity C₂ of barium ions in the pretreated solution by using a barium ion electrode-sensing analyzer, and calculating the sulfate ion content in the saline soil to be tested according to Equation 1:

$${C}_{sulfate ion} = 9.6 q ( {V}_{2} {C}_{1} - ( {V}_{1} + {V}_{2} ) {C}_{2} ) / {V}_{1} ; \qquad Equation 1$$

wherein
in Equation 1, C_{sulfate ion} represents the sulfate ion content in the saline soil to be tested, in wt %;
q is the mass ratio of the saline soil to be tested to the water, with no unit;
V₁ is a volume of the saline soil leachate, in mL;
V₂ is a volume of the barium chloride solution, in mL;
C₁ is a molarity of the barium chloride solution, in mol/L; and
C₂ is a molarity of the barium ions in the pretreated solution, in mol/L.

In some embodiments, determining a chloride ion content in the saline soil to be tested includes the following steps:
mixing the saline soil to be tested with water, and conducting solid-liquid separation to obtain the saline soil leachate, wherein a mass ratio of the saline soil to be tested to the water is denoted as q;
taking the saline soil leachate with a volume of V₃, and measuring a molarity C₃ of chloride ions in the saline soil leachate by using a chloride ion electrode-sensing analyzer; and
calculating the chloride ion content in the saline soil to be tested according to Equation 2:

$${C}_{chloride ion} = 3.55 {qC}_{3} ; \qquad Equation 2$$

wherein
in Equation 2, C_{chloride ion} represents the chloride ion content in the saline soil to be tested, in wt %;
q is the mass ratio of the saline soil to be tested to the water, with no unit; and
C₃ represents the molarity of the chloride ions measured by the chloride ion electrode-sensing analyzer, in mol/L.

In some embodiments, evaluating salinization degree of the saline soil to be tested includes
(1) under the condition that the saline soil to be tested is taken from a fine-grained soil layer,
1) under the condition that the saline soil to be tested is the chlorine saline soil or the sub-chlorine saline soil,
evaluating the saline soil to be tested as weak saline soil under the condition that the TSSC is greater than or equal to 0.3 wt % and less than 1 wt %;
evaluating the saline soil to be tested as moderate saline soil under the condition that the TSSC is greater than or equal to 1 wt % and less than 5 wt %;
evaluating the saline soil to be tested as strong saline soil under the condition that the TSSC is greater than or equal to 5 wt % and less than 8 wt %; and
evaluating the saline soil to be tested as excessive saline soil under the condition that the TSSC is greater than or equal to 8 wt %;
2) under the condition that the saline soil to be tested is the sulfite saline soil or the sulfate saline soil,
evaluating the saline soil to be tested as weak saline soil under the condition that the TSSC is greater than or equal to 0.3 wt % and less than 0.5 wt %;
evaluating the saline soil to be tested as moderate saline soil under the condition that the TSSC is greater than or equal to 0.5 wt % and less than 2 wt %;
evaluating the saline soil to be tested as strong saline soil under the condition that the TSSC is greater than or equal to 2 wt % and less than 5 wt %; and
evaluating the saline soil to be tested as excessive saline soil under the condition that the TSSC is greater than or equal to 5 wt %; and
(2) under the condition that the saline soil to be tested is taken from a coarse-grained soil layer,
1) under the condition that the saline soil to be tested is the chlorine saline soil or the sub-chlorine saline soil,
evaluating the saline soil to be tested as weak saline soil under the condition that the TSSC is greater than or equal to 2 wt % and less than 5 wt %;
evaluating the saline soil to be tested as moderate saline soil under the condition that the TSSC is greater than or equal to 5 wt % and less than 8 wt %;
evaluating the saline soil to be tested as strong saline soil under the condition that the TSSC is greater than or equal to 8 wt % and less than 10 wt %; and
evaluating the saline soil to be tested as excessive saline soil under the condition that the TSSC is greater than or equal to 10 wt %; and
2) under the condition that the saline soil to be tested is the sulfite saline soil or the sulfate saline soil,
evaluating the saline soil to be tested as weak saline soil under the condition that the TSSC is greater than or equal to 0.5 wt % and less than 1.5 wt %;
evaluating the saline soil to be tested as moderate saline soil under the condition that the TSSC is greater than or equal to 1.5 wt % and less than 3 wt %;
evaluating the saline soil to be tested as strong saline soil under the condition that the TSSC is greater than or equal to 3 wt % and less than 6 wt %; and
evaluating the saline soil to be tested as excessive saline soil under the condition that the TSSC is greater than or equal to 6 wt %.

In some embodiments, the fine-grained soil layer has a particle size of less than or equal to 0.075 mm, and the coarse-grained soil layer has a particle size of greater than 0.075 mm and less than or equal to 60 mm.

The present disclosure provides a method for determining a TSSC in a saline soil, including the following steps: mixing the saline soil to be tested with water, and conducting solid-liquid separation to obtain a saline soil leachate; and measuring a refractive index of the saline soil leachate by using a calibrated refractometer, and obtaining the TSSC of the saline soil to be tested according to a predetermined standard curve and the refractive index of the saline soil leachate; wherein the TSSC is expressed by a mass percentage content. In the present disclosure, based on a principle for measuring a refractive index, as well as a relationship that the refractive index is directly proportional to a solution concentration, the TSSC is rapidly determined through the measured refractive index and a linear relationship between the refractive index of a saline soil leachate and the TSSC. The results of the examples show that under the condition that a mass ratio of the saline soil to the water is 1:1, the standard curve is represented by a function: y=602.8x−803.4, and a correlation coefficient R² is 0.998; under the condition that a mass ratio of the saline soil to the water is 1:3, the standard curve is represented by a function: y=1958.5x−2610.7, the correlation coefficient R² is 0.999; and under the condition that a mass ratio of the saline soil to the water is 1:5, the standard curve is represented by a function: y=3290.7x−4386.6, the correlation coefficient R² is 0.997, wherein in these functions, x represents the refractive index, and y represents the TSSC, in %. The above standard curves have a limit of detection (LOD) of 0.3% and a linear detection range of 0.3% to 45%. In the present disclosure, the determination method has simple and fast operations, and is suitable for rapid determination of the TSSC in the saline soil at a construction site.

The present disclosure further provides a method for evaluating a soil type and a salinization degree of a saline soil. In the present disclosure, the TSSC is quickly determined by using a portable refractometer; and contents of chloride ions and sulfate ions are quickly determined by using a portable chloride ion electrode-sensing analyzer and a portable barium ion electrode-sensing analyzer, respectively. In this way, the rapid determination of TSSC and the evaluation of salinization degree in the saline soil are realized at a construction site. Overall test process in the method takes not more than 1 h, which greatly improves a speed of evaluating the soil type and the salinization degree of the saline soil.

### BRIEF DESCRIPTION OF THE DRAWINGS

FIG. 1 shows a standard curve obtained when a mass ratio of the saline soil to water is 1:1.

FIG. 2 shows a standard curve obtained when a mass ratio of the saline soil to water is 1:3.

FIG. 3 shows a standard curve obtained when a mass ratio of the saline soil to water is 1:5.

### DETAILED DESCRIPTION OF THE EMBODIMENTS

The present disclosure provides a method for determining a TSSC in a saline soil, including the following steps:
mixing a saline soil to be tested with water, and conducting solid-liquid separation to obtain a saline soil leachate; and
measuring a refractive index of the saline soil leachate by using a calibrated refractometer, and obtaining the TSSC of the saline soil to be tested according to a predetermined standard curve and the refractive index of the saline soil leachate;
wherein the TSSC is expressed by a mass percentage content; and
the standard curve is a linear relationship curve between the TSSC and the refractive index.

In some embodiments of the present disclosure, the saline soil to be tested is sampled from a fine-grained soil layer or a coarse-grained soil layer. In some embodiments, the fine-grained soil layer has a particle size of less than or equal to 0.075 mm. In some embodiments, the coarse-grained soil layer has a particle size of greater than 0.075 mm and less than or equal to 60 mm.

In some embodiments of the present disclosure, 10 g to 50 g, preferably 20 g to 40 g of the saline soil to be tested is sampled. In some embodiments, the saline soil to be tested is sieved. In some embodiments, the saline soil to be tested is sieved by using a sieve with a pore size of 1 mm.

In some embodiments of the present disclosure, the water is distilled water free of carbon dioxide. In some embodiments, a mass ratio of the saline soil to the water is in a range of 1:(1-5), and preferably 1:5.

In some embodiments of the present disclosure, the mixing is conducted by stirring or shaking. In some embodiments, the mixing is conducted for 3 min to 5 min. In some embodiments, the solid-liquid separation is conducted by filtration.

In some embodiments of the present disclosure, the refractometer is a digital-display probe-type refractometer. In some embodiments, the refractometer is calibrated by process including: cleaning a test lens of a probe of the refractometer with distilled water, and pressing a calibration key to calibrate the refractometer, under the condition that the refractive index of distilled water is read as 1.3329±0.0001, the calibration of the refractometer is finished. In some embodiments, after the calibration, the test lens of the probe is washed again with distilled water.

In the present disclosure, the standard curve is obtained by a process including the following steps:
providing a series of saline soil leachates with a concentration gradient having known TSSC; and
measuring a refractive index of each of the saline soil leachates having known TSSC by a calibrated refractometer, and obtaining the corresponding refractive index of each of the saline soil leachates with a concentration gradient with known TSSC; and plotting the standard curve with the refractive index as an abscissa and the TSSC as an ordinate.

In some embodiments of the present disclosure, a series of the saline soil leachates with a concentration gradient having known TSSC are prepared by a process including the following steps:
preparing the saline soil leachates according to the above process from not less than 50 saline soils with different salinization degrees and soil types; determining the TSSC of the saline soils according to a mass method stipulated in “Test Methods of Soils for Highway Engineering” (JTG 3430-2020), to obtain the TSSC of the saline soil.

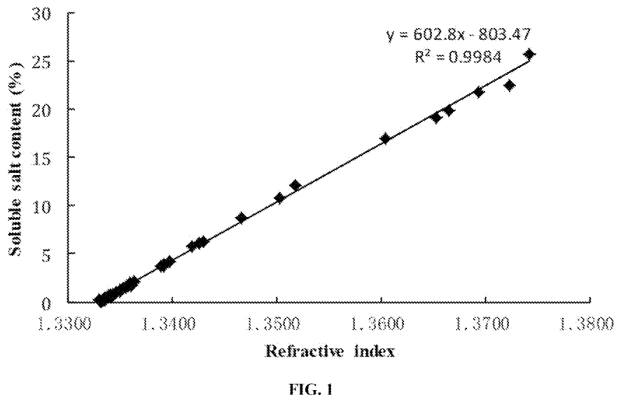

In some embodiments of the present disclosure, under the condition that a mass ratio of the saline soil to water is 1:1, the standard curve is represented by a function: y=602.8x−803.4, and the correlation coefficient R² is 0.998; the standard curve is shown in FIG. 1.

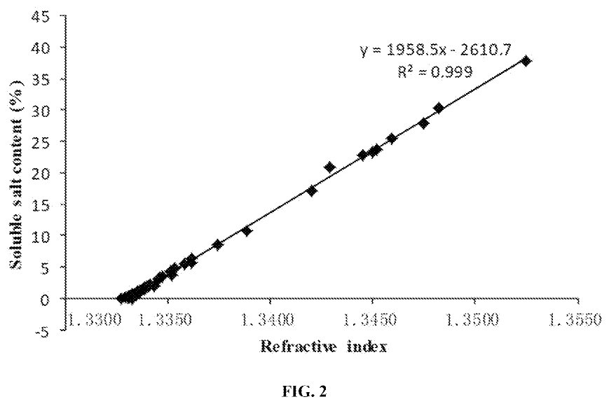

In some embodiments of the present disclosure, under the condition that a mass ratio of the saline soil to water is 1:3, the standard curve is represented by a function: y=1958.5x−2610.7, and the correlation coefficient R² is 0.999; the standard curve is shown in FIG. 2.

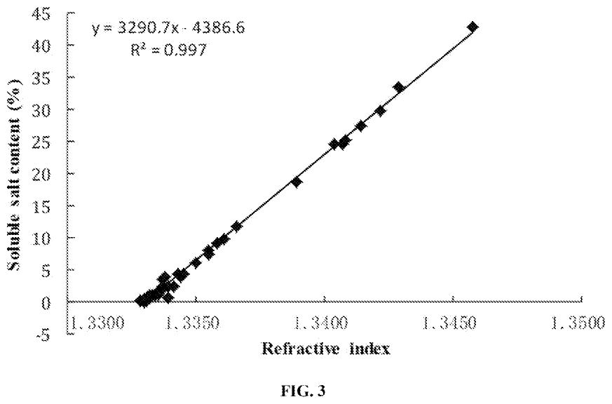

In some embodiments of the present disclosure, under the condition that a mass ratio of the saline soil to water is 1:5, the standard curve is represented by a function: y=3290.7x−4386.6, and the correlation coefficient R² is 0.997; the standard curve is shown in FIG. 3.

The present disclosure further provides a method for evaluating a soil type and a salinization degree of a saline soil, including the following steps:
determining a TSSC of the saline soil to be tested by the method as described in above technical solutions;
adding a barium chloride solution to a saline soil leachate, and determining a sulfate ion content in the saline soil to be tested by using a barium ion electrode-sensing analyzer;
determining a chloride ion content in the saline soil to be tested by using a chloride ion electrode-sensing analyzer;
calculating a ratio k of the chloride ion content to the sulfate ion content;
evaluating the soil type of the saline soil to be tested according to the ratio k, wherein under the condition that the ratio k is greater than 2, the soil type of the saline soil to be tested is chlorine saline soil; under the condition that the ratio k is not less than 1 and not more than 2, the soil type of the saline soil to be tested is sub-chlorine saline soil; under the condition that the ratio k is larger than or equal to 0.3 and less than 1, the soil type of the saline soil to be tested is sulfite saline soil; and under the condition that the ratio k is less than 0.3, the soil type of the saline soil to be tested is sulfate saline soil; and
evaluating salinization degree of the saline soil to be tested with the TSSC and the soil type of the saline soil to be tested according to the “Code for Highway Engineering Geological Investigation” (JTG C20-2011).

In the present disclosure, a method for determining the TSSC of the saline soil to be tested is the same as above, and will not be repeated here.

In the present disclosure, a sulfate ion content in the saline soil to be tested is determined by using a barium ion electrode-sensing analyzer. In some embodiments, determining a sulfate ion content in the saline soil to be tested includes the following steps:
mixing the saline soil to be tested with water, and conducting solid-liquid separation to obtain the saline soil leachate, wherein a mass ratio of the saline soil to be tested to the water is denoted as q;
adding 5 to 10 drops of a hydrochloric acid solution into the saline soil leachate with a volume of V₁, heating to boiling, adding a barium chloride solution with a volume of V₂ and a molarity of C₁ thereto, reheating a resulting mixture to boiling, and allowing to stand and cooling, to obtain a pretreated solution; and
measuring a molarity C₂ of barium ions in the pretreated solution by using a barium ion electrode-sensing analyzer, and calculating the sulfate ion content in the saline soil to be tested according to Equation 1:

$${C}_{sulphate ion} = 9.6 q ( {V}_{2} {C}_{1} - ( {V}_{1} + {V}_{2} ) {C}_{2} ) / {V}_{1} ; \qquad Equation 1$$

in Equation 1, C_{sulfate ion} represents the sulfate ion content in the saline soil to be tested, in wt %;
q is the mass ratio of the saline soil to be tested to the water, with no unit;
V₁ is the volume of the saline soil leachate, in mL;
V₂ is the volume of the barium chloride solution, in mL;
C₁ is the molarity of the barium chloride solution, in mol/L; and
C₂ is the molarity of the barium ions in the pretreated solution, in mol/L.

In the present disclosure, a process for preparing the saline soil leachate is the same as above, and will not be repeated here. In some embodiments, the saline soil leachate has a V₁ volume of 20 mL to 50 mL, and preferably 30 mL to 40 mL. In some embodiments, the hydrochloric acid solution has a concentration of 3 mol/L.

In some embodiments of the present disclosure, the barium chloride solution has a concentration C₁ of 0.02 mol/L to 0.05 mol/L, and preferably 0.03 mol/L to 0.04 mol/L. In some embodiments, the barium chloride solution has a V₂ volume of 10 mL to 20 mL, and preferably 15 mL.

In some embodiments of the present disclosure, reheating the resulting mixture to boiling is conducted for 3 min to 5 min, and preferably 4 min. In some embodiments, allowing to stand and cooling is conducted until room temperature.

In the present disclosure, the barium ion electrode-sensing analyzer is provided with a barium ion composite electrode. In some embodiments, measuring the molarity of the barium ions in the pretreated solution by using a barium ion electrode-sensing analyzer includes
immersing the barium ion composite electrode in a barium chloride standard solution of 1,000 ppm, and preforming pretreatment for 10 min to 20 min until the analyzer has a stable reading; and
after the reading is obtained, taking out the barium ion composite electrode, washing the barium ion composite electrode by spraying deionized water, and drying with absorbent paper.

In the present disclosure, the barium ion concentration read by the barium ion electrode-sensing analyzer is remaining barium ion concentration after a reaction between the added barium chloride solution and the sulfate ions. The barium ions consumed has equivalent concentration to that of sulfate ions; as such, the sulfate ion content in the saline soil is calculated according to the Equation:

$${C}_{sulfate ion} ( mmol / kg ) = 1000 q ( {V}_{2} {C}_{1} - ( {V}_{1} + {V}_{2} ) {C}_{2} ) / {V}_{1} ,$$

With respective to the weight content, the above equation is converted into Equation 1:

$${C}_{sulfate ion} = 9.6 q ( {V}_{2} {C}_{1} - ( {V}_{1} + {V}_{2} ) {C}_{2} ) / {V}_{1} . \qquad Equation 1$$

In the present disclosure, determining a chloride ion content in the saline soil to be tested includes the following steps:
mixing the saline soil to be tested with water, and conducting solid-liquid separation to obtain the saline soil leachate, wherein a mass ratio of the saline soil to be tested to the water is denoted as q;
taking the saline soil leachate with a volume of V₃, and measuring a molarity C₃ of chloride ions in the saline soil leachate by using a chloride ion electrode-sensing analyzer; and
calculating the chloride ion content in the saline soil to be tested according to Equation 2:

$${C}_{chloride ion} = 3.55 {qC}_{3} ; \qquad Equation 2$$

in Equation 2, C_{chloride ion} represents the chloride ion content in the saline soil to be tested, in wt %;
q is the mass ratio of the saline soil to be tested to the water, with no unit;
C₃ represents the molarity of the chloride ions measured by the chloride ion electrode-sensing analyzer, in mol/L.

In the present disclosure, a process for preparing the saline soil leachate is the same as above, and will not be repeated here.

In some embodiments, the saline soil leachate has a V₃ volume of 20 mL to 50 mL, and preferably 40 mL to 50 mL.

In some embodiments of the present disclosure, the chloride ion electrode-sensing analyzer is provided with a chloride ion composite electrode. In some embodiments, before test with the chloride ion electrode-sensing analyzer, the chloride ion composite electrode is calibrated. In some embodiments, the chloride ion composite electrode is calibrated by a process including the following steps:
pressing a “calibration key” of the chloride ion electrode-sensing analyzer, putting the chloride ion composite electrode into distilled water, and allowing to stand; after a reading is stable, pressing a “confirmation key” to calibrate a zero point; putting the chloride ion composite electrode into a 1,000 ppm chloride ion standard solution and allowing to stand; after the reading is stable, pressing the “confirmation key”; after the reading of 1,000 ppm is successfully displayed, terminating the calibration with chloride ion.

In some embodiments of the present disclosure, after the molarity of the chloride ions in the saline soil leachate is tested with the chloride ion electrode-sensing analyzer, the chloride ion composite electrode is taken out, washed by spraying deionized water, dried with absorbent paper, and covered with an electrode protective cap.

In the present disclosure, the chloride ion concentration read by the chloride ion electrode-sensing analyzer is the molarity. As such, the chloride ion content in the saline soil is calculated according to the equation: C_{chloride ion} (mmol/kg)=1000qC₃;

With respective to weight content, the above equation is converted into Equation 2:

$${C}_{chloride ion} = 3.55 {qC}_{3} . \qquad Equation 2$$

In the present disclosure, a ratio k of the chloride ion content to the sulfate ion content is calculated; under the condition that the ratio k is larger than 2, the soil type of the saline soil to be tested is chlorine saline soil; under the condition that the ratio k is not less than 1 and not more than 2, the saline soil to be tested is sub-chlorine saline soil; under the condition that the ratio k is larger than or equal to 0.3 and less than 1, the soil type of the saline soil to be tested is sulfite saline soil; and under the condition that the ratio k is less than 0.3, the soil type of the saline soil to be tested is sulfate saline soil.

In the present disclosure, the evaluation of the soil type of the saline soil to be tested is shown in Table 1.

TABLE 1
Evaluation of soil type of the saline soil to be tested

| Type of saline soil | Cl⁻/SO₄²⁻ ratio |
|---|---|
| chlorine saline soil | >2 |
| sub-chlorine saline soil | 1-2 |
| Sulfite saline soil | 0.3-1 |
| Sulfate saline soil | <0.3 |

After the soil type of the saline soil to be tested is obtained, the salinization degree of the saline soil to be tested is evaluated according to the “Code for Highway Engineering Geological Investigation” (JTG C20-2011). In some embodiments of the present disclosure, evaluation of the salinization degree of the saline soil is shown in Table 2.

TABLE 2
Evaluation of the salinization degree of the saline soil

| | | | Average salt content of | |
|---|---|---|---|---|
| | | | coarse-grained soil passing through | |
| | Average salt content of fine-grained soil | | a 10 mm sieve (%) | |
| | layer (in percentages by mass, %) | | Chlorine saline | Sulfite saline |
| | Chlorine saline soil | Sulfite saline soil | soil | soil |
| Salinization | Sub-chlorine saline | Sulfate saline | Sub-chlorine | Sulfate saline |
| degree | soil | soil | saline soil | soil |
| Weak saline | 0.3-1 | 0.3-0.5 | 2.0-5.0 | 0.5-1.5 |
| soil | | | | |
| Moderate | 1.0-5.0 | 0.5-2.0 | 5.0-8.0 | 1.5-3.0 |
| saline soil | | | | |
| Strong saline | 5.0-8.0 | 2.0-5.0 | 8.0-10.0 | 3.0-6.0 |
| soil | | | | |
| Excessive | >8.0 | >5.0 | >10.0 | >6.0 |
| saline soil | | | | |

The method for determining a TSSC in a saline soil, and the method for evaluating a soil type and a salinization degree of saline soil according to the present disclosure are described in detail below with reference to the examples, but these examples shall not be understood as a limitation to the scope of the present disclosure.

### Example 1

A saline soil sample from Jiayuguan highway construction project (China) was tested according to the following procedures:

Step I, preparation of a saline soil leachate:

A saline soil sample from the Jiayuguan highway construction project was dried and passed through a 1 mm sieve. 50 g of the saline soil sample passing through the 1 mm sieve was put into a dry conical flask with a stopper, and carbon dioxide-free distilled water was added thereto (a mass ratio of the saline soil to water being 1:5). The conical flask was closed with the stopper. After shaking vigorously by hand for 5 min, a saline soil sample suspension was obtained and immediately poured into an integrated filter device for preparing the saline soil sample leachate. A piston of the device was pressed to pressurize, and a clear leachate was obtained from the filter device.

Step II, determination of a TSSC of the saline soil:
(1) A digital-display probe-type refractometer was started, a test lens of a probe was cleaned with distilled water. The refractometer was calibrated with distilled water as follows: the probe was immersed in the distilled water (ensuring that the lens was completely submerged in the liquid); a calibration key was pressed to calibrate the refractometer, and the refractive index of the distilled water was read as 1.3330 after the calibration.
(2) After the calibration, the lens was cleaned again with distilled water, and the probe was immersed in the soil sample leachate to start the test, and a refractive index was read as 1.3336.
(3) The TSSC was calculated by plugging the refractive index into a function: y=3290.7x−4386.6 (representing a relationship between the refractive index and the TSSC under the condition that a mas ratio of the saline soil to the water was 1:5), obtaining a TSSC of 1.9%.

Step III, determination of a sulfate ion content in the saline soil:
(1) 25 mL (V₁) of the saline soil leachate obtained in step I was added to a conical flask, and 5 drops of 3 mol/L HCl was added thereto. They were heated to boiling, and 20 mL of a barium chloride solution at a concentration of 0.05 mol/L (C₁) was accurately added thereto by a pipettor. The resulting mixture was boiled slightly for 3 min, allowed to stand and cooled to room temperature.
(2) A barium ion composite electrode was immersed in a barium chloride standard solution of 1,000 ppm, and a pretreatment was performed for 10 min until the analyzer had a stable reading.
(3) The barium ion composite electrode was taken out, washed by spraying deionized water, and dried with absorbent paper.
(4) The leachate treated in step (1) was transferred to a plastic beaker, and the barium ion composite electrode was placed therein for measuring. After 3 min, a barium ion concentration was read as 0.013 mol/L. After the test, the electrode was washed by spraying deionized water, dried with absorbent paper, and covered with a protective cap.
(5) Calculation: the barium ion concentration read by the barium ion electrode-sensing analyzer was a remaining barium ion concentration after a reaction between the added barium chloride solution and the sulfate ions. The barium ions consumed had equivalent concentration to that of sulfate ions; as such, the sulfate ion content in the saline soil is calculated as follows:

$${SO 4}^{2 -} ( mmol / kg ) = 1000 \times 5 \times ( 20 \times 0 \text{.05} - ( 25 + 20 ) \times 0.0 \; 13 ) \div 25 = 83 mmol / kg ;$$

$${SO 4}^{2 -} ( \% ) = 9.6 \times 5 \times ( 20 \times 0.05 - ( 25 + 20 ) \times 0.0 \; 13 ) \div 25 = 0.8 \% .$$

Step IV, determination of a chloride ion content in the saline soil:
(1) Calibration of a chloride ion composite electrode: a “calibration key” of a chloride ion electrode-sensing analyzer was pressed, the chloride ion composite electrode was put into distilled water, and allowed to stand; after a reading was stable, a “confirmation key” was pressed to calibrate a zero point. The chloride ion composite electrode was put into a 1,000 ppm chloride ion standard solution and allowed to stand; after the reading was stable, the “confirmation key” was pressed; the calibration with chloride ion was terminated when the reading of 1,000 ppm was successfully displayed.
(2) Measurement of chloride ions: 25 mL (V₃) of the saline soil leachate was placed into a plastic beaker or centrifuge tube, and the chloride ion composite electrode was immersed therein for determination. After a reading was stable, a molarity of the chlorine ions was read as 0.012 mol/L. The chloride ion composite electrode was taken out, washed by spraying deionized water, dried with absorbent paper, and covered with an electrode protective cap.
(3) Calculation: a chloride ion concentration read was a molarity, and the chloride ion content in the saline soil was calculated as follows:

$${Cl}^{-} ( mmol / kg ) = 1000 \times 5 \times 0.012 = 60 mmol / kg ;$$

$${Cl}^{-} ( \% ) = 3.55 \times 5 \times 0.012 = 0.21 \% .$$

According to the “Code for Highway Engineering Geological Investigation” (JTG C20-2011), the TSSC, the chloride ion content, and sulfate ion content were plugged, and thus Cl⁻(mmol/kg)/SO4²⁻ (mmol/kg)=60 (mmol/kg)/83 (mmol/kg)=0.7. This saline soil was evaluated as sulfite saline soil with a TSS of 1.9% according to Table 1, as well as moderate saline soil according to Table 2. In summary, this saline soil was moderate sulfite saline soil.

### Example 2

A saline soil sample from Lanzhou highway construction project (China) was tested according to the following procedures:

Step I, preparation of a saline soil leachate:

A saline soil sample from the Lanzhou highway construction project was dried and passed through a 1 mm sieve. 50 g of the saline soil sample passing through the 1 mm sieve was put into a dry conical flask with a stopper, and carbon dioxide-free distilled water was added thereto (a mass ratio of the saline soil to the water being 1:3). The conical flask was closed with the stopper. After shaking vigorously by hand for 3 min, a saline soil sample suspension was obtained and immediately poured into an integrated filter device for preparing the saline soil sample leachate. A piston of the device was pressed to pressurize, and a clear leachate was obtained from the filter device.

Step II, determination of a TSSC of the saline soil:
(1) A digital-display probe-type refractometer was started, and a test lens of a probe was cleaned with distilled water. The refractometer was calibrated with distilled water as follows: the probe was immersed in the distilled water (ensuring that the lens was completely submerged in the liquid); a calibration key was pressed to calibrate the refractometer, and the refractive index of the distilled water was read as 1.3329 after the calibration.
(2) After the calibration, the lens was cleaned again with distilled water, and the probe was immersed in the soil sample leachate to start the test, and a refractive index was read as 1.3333.
(3) The TSSC was calculated by plugging the refractive index into a function: y=1958.5x−2610.7 (representing a relationship between the refractive index and the TSSC under the condition that a mass ratio of the saline soil to the water was 1:3), obtaining a TSSC of 0.6%.

Step III, determination of a sulfate ion content in the saline soil:
(1) 25 mL (V₁) of the saline soil leachate obtained in step I was added to a conical flask, and 6 drops of 3 mol/L HCl was added thereto. They were heated to boiling, and 20 mL of a barium chloride solution at a concentration of 0.02 mol/L (C₁) was accurately added thereto by a pipettor. The resulting mixture was boiled slightly for 5 min, allowed to stand and cooled to room temperature.
(2) A barium ion composite electrode was immersed in a barium chloride standard solution of 1,000 ppm and a pretreatment was performed for 20 min until the analyzer had a stable reading.
(3) The barium ion composite electrode was taken out, washed by spraying with deionized water, and dried with absorbent paper.
(4) The leachate treated in step (1) was transferred to a plastic beaker, and the barium ion composite electrode was placed therein for measuring. After 3 min, a barium ion concentration was read as 0.008 mol/L. After the test, the electrode was washed by spraying deionized water, dried with absorbent paper, and covered with a protective cap.
(5) Calculation: the barium ion concentration read by the barium ion electrode-sensing analyzer was a remaining barium ion concentration after a reaction between the added barium chloride solution and the sulfate ions. The barium ions consumed had equivalent concentration to that of sulfate ions; as such, the sulfate ion content in the saline soil was calculated as follows:

$${SO 4}^{2 -} ( mmol / kg ) = 1000 \times 5 \times ( 20 \times 0 \text{.02} - ( 25 + 20 ) \times 0.0 \; 08 ) \div 25 = 8 mmol / kg ;$$

$$SO {4}^{2 -} ( \% ) = 9.6 \times 5 \times ( 20 \times 0.02 - ( 25 + 20 ) \times 0.008 ) \div 25 = 0.08 \% .$$

Step IV, determination of a chloride ion content in the saline soil:
(1) Calibration of a chloride ion composite electrode: a “calibration key” of a chloride ion electrode-sensing analyzer was pressed, the chloride ion composite electrode was put into distilled water, and allowed to stand; after a reading was stable, a “confirmation key” was pressed to calibrate a zero point. The chloride ion composite electrode was put into a 1,000 ppm chloride ion standard solution and allowed to stand; after the reading was stable, the “confirmation key” was pressed; the calibration with chloride ion was terminated when the reading of 1,000 ppm was successfully displayed.
(2) Measurement of chloride ions: 50 mL (V₃) of the saline soil leachate was placed into a plastic beaker or centrifuge tube, and the chloride ion composite electrode was immersed therein for determination. After a reading was stable, a molarity of the chlorine ions was read as 0.006 mol/L. The chloride ion composite electrode was taken out, washed by spraying deionized water, dried with absorbent paper, and covered with an electrode protective cap.
(3) Calculation: a chloride ion concentration read was a molarity, and the chloride ion content in the saline soil was calculated as follows:

$${Cl}^{-} ( mmol / kg ) = 1000 \times 5 \times 0.006 = 30 mmol / kg ;$$

$${Cl}^{-} ( \% ) = 3.55 \times 5 \times 0.006 = 0.11 \% .$$

Step IV, evaluation of a soil type and a salinization degree of saline soil by calculation:

According to the “Code for Highway Engineering Geological Investigation” (JTG C20-2011), the TSSC, the chloride ion content, and sulfate ion content were plugged, and thus Cl⁻ (mmol/kg)/SO4²⁻ (mmol/kg)=30 (mmol/kg)/8 (mmol/kg)=3.75. This saline soil was evaluated as chlorine saline soil with a TSSC of 0.6% according to Table 1, as well as weak saline soil according to Table 2. In summary, this saline soil was weak chlorine saline soil.

### Example 3

A saline soil sample from Jiuquan highway construction project (China) was tested according to the following procedures:

Step I, preparation of a saline soil leachate:

A saline soil sample near the Jiuquan highway construction project was dried and passed through a 1 mm sieve. 25 g of the saline soil sample passing through the 1 mm sieve was put into a dry conical flask with a stopper, and carbon dioxide-free distilled water was added thereto (a mass ratio of the saline soil to the water being 1:5). The conical flask was closed with the stopper. After shaking vigorously by hand for 5 min, a saline soil sample suspension was obtained and immediately poured into an integrated preparation filter device for preparing the saline soil sample leachate. A piston of the device was pressed to pressurize, and a clear leachate was obtained from the filter device.

Step II, determination of a TSSC of saline soil:
(1) A digital-display probe-type refractometer was started, and a test lens of a probe was cleaned with distilled water. The refractometer was calibrated with distilled water as follows: the probe was immersed in the distilled water (ensuring that the lens was completely submerged in the liquid); a calibration key was pressed to calibrate the refractometer, and the refractive index of the distilled water was read as 1.3330 after the calibration.
(2) After the calibration, the lens was cleaned again with distilled water, and the probe was immersed in the soil sample leachate to start the test, and a refractive index was read as 1.3339.
(3) The TSSC was calculated by plugging the refractive index into a function: w=3290.7n−4386.6 (representing a relationship between the refractive index and the TSSC under the condition that a mass ratio of the saline soil to the water was 1:5), obtaining a TSSC of 2.9%.

Step III, determination of a sulfate ion content in the saline soil:
(1) 20 mL (V₁) of the saline soil leachate obtained in step I was added to a conical flask, and 6 drops of 3 mol/L HCl was added thereto. They were heated to boiling, and 15 mL of a barium chloride solution at a concentration of 0.05 mol/L (C₁) was accurately added thereto by a pipettor. The resulting mixture was boiled slightly for 5 min, allowed to stand and cooled to room temperature.
(2) A barium ion composite electrode was immersed in a barium chloride standard solution of 1,000 ppm, and a pretreatment was performed for 10 min until the analyzer had a stable reading.
(3) The barium ion composite electrode was taken out, washed by spraying deionized water, and dried with absorbent paper.
(4) The leachate treated in step (1) was transferred to a plastic beaker, and the barium ion composite electrode was placed therein for measuring. After 3 min, a barium ion concentration was read as 0.006 mol/L. After the test, the electrode was washed by spraying deionized water, dried with absorbent paper, and covered with a protective cap.
(5) Calculation: the barium ion concentration read by the barium ion electrode-sensing analyzer was a remaining barium ion concentration after a reaction between the added barium chloride solution and the sulfate ions. The barium ions consumed had equivalent concentration to that of sulfate ions consumed; as such, the sulfate ion content in the saline soil was calculated as follows:

$$SO {4}^{2 -} ( mmol / kg ) = 1000 \times 5 \times ( 15 \times 0.05 - ( 25 + 20 ) \times 0.006 ) \div 20 = 135 mmol / kg ;$$

$$SO {4}^{2 -} ( \% ) = 9.6 \times 5 \times ( 15 \times 0.05 - ( 20 + 15 ) \times 0.0 \; 06 ) \div 20 = 1.3 \% .$$

Step IV, determination of a chloride ion content in the saline soil:
(1) Calibration of a chloride ion composite electrode: a “calibration key” of a chloride ion electrode-sensing analyzer was pressed, the chloride ion composite electrode was put into distilled water, and allowed to stand; after a reading was stable, a “confirmation key” was pressed to calibrate a zero point. The chloride ion composite electrode was put into a 1,000 ppm chloride ion standard solution and allowed to stand; after the reading was stable, the “confirmation key” was pressed; the calibration with chloride ion was terminated when the reading of 1,000 ppm was successfully displayed.
(2) Measurement of chloride ions: 50 mL (V₃) of the saline soil leachate was placed into a plastic beaker or centrifuge tube, and the chloride ion composite electrode was immersed therein for determination. After a reading was stable, a molarity of the chlorine ions was read as 0.033 mol/L. The chloride ion composite electrode was taken out, washed by spraying deionized water, dried with absorbent paper, and covered with an electrode protective cap.
(3) Calculation: a chloride ion concentration read was a molarity, and the chloride ion content in the saline soil was calculated as follows:

$${Cl}^{-} ( mmol / kg ) = 1000 \times 5 \times 0.033 = 165 mmol / kg ;$$

$${Cl}^{-} ( \% ) = 3.55 \times 5 \times 0. 033 = 0.59 \% .$$

Step IV, Evaluation of a soil type and a salinization degree of saline soil by calculation:

According to the “Code for Highway Engineering Geological Investigation” (JTG C20-2011), the TSSC, the chloride ion content, and sulfate ion content were plugged, and thus Cl⁻ (mmol/kg)/SO4²⁻ (mmol/kg)=165 (mmol/kg)/135 (mmol/kg)=1.2. This saline soil was determined as sub-chlorine saline soil with a TSSC of 2.9% according to Table 1, as well as moderate saline soil according to Table 2. In summary, this saline soil was moderate sub-chlorine saline soil.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for determining a total soluble salt content (TSSC) in a saline soil, comprising the steps of
mixing a saline soil to be tested with water, and conducting solid-liquid separation to obtain a saline soil leachate; and
measuring a refractive index of the saline soil leachate by using a calibrated refractometer, and obtaining the TSSC of the saline soil to be tested according to a predetermined standard curve and the refractive index of the saline soil leachate measured;
wherein the TSSC is expressed by a mass percentage content; and
the standard curve is a linear relationship curve between the TSSC and the refractive index.

2. The method as claimed in claim 1, wherein the water is distilled water free of carbon dioxide.

3. The method as claimed in claim 1, wherein a mass ratio of the saline soil to be tested to the water is in a range of 1:(1-5).

4. The method as claimed in claim 1, wherein the standard curve is obtained by a process comprising the steps of
providing a series of saline soil leachates with a concentration gradient having known TSSC, as standard samples to be tested; and
measuring refractive indexes of the standard samples to be tested, and plotting the standard curve with the refractive index as an abscissa and the soluble salt content as an ordinate.

5. The method as claimed in claim 1, wherein the refractometer is a digital-display probe-type refractometer.

6. A method for evaluating a soil type and a salinization degree of a saline soil, comprising the steps of
determining a total soluble salt content (TSSC) of the saline soil to be tested by the method as claimed in claim 1;
adding a barium chloride solution to a saline soil leachate, and determining a sulfate ion content in the saline soil to be tested by using a barium ion electrode-sensing analyzer;
determining a chloride ion content in the saline soil to be tested by using a chloride ion electrode-sensing analyzer;
calculating a ratio k of the chloride ion content to the sulfate ion content;
evaluating the soil type of the saline soil to be tested according to the ratio k, wherein under the condition that the ratio k is greater than 2, the soil type of the saline soil to be tested is chlorine saline soil; under the condition that the ratio k is not less than 1 and not more than 2, the soil type of the saline soil to be tested is sub-chlorine saline soil; under the condition that the ratio k is larger than or equal to 0.3 and less than 1, the soil type of the saline soil to be tested is sulfite saline soil; and under the condition that the ratio k is less than 0.3, the soil type of the saline soil to be tested is sulfate saline soil; and
evaluating the salinization degree of the saline soil to be tested with the TSSC and the soil type of the saline soil to be tested according to the “code for highway engineering geological investigation”.

7. The method as claimed in claim 6, wherein determining a sulfate ion content in the saline soil to be tested comprises
mixing the saline soil to be tested with water, and conducting solid-liquid separation to obtain the saline soil leachate, wherein a mass ratio of the saline soil to be tested to the water is denoted as q;
adding 5 to 10 drops of a hydrochloric acid solution into the saline soil leachate with a volume of V₁, heating to boiling, adding a barium chloride solution with a volume of V₂ and a molarity of C₁ thereto, reheating a resulting mixture to boiling, and allowing to stand and cooling, to obtain a pretreated solution; and
measuring a molarity C₂ of barium ions in the pretreated solution by using a barium ion electrode-sensing analyzer, and calculating the sulfate ion content in the saline soil to be tested according to Equation 1:
$${C}_{sulfacte ion} = 9.6 q ( {V}_{2} {C}_{1} - ( {V}_{1} + {V}_{2} ) {C}_{2} ) / {V}_{1} ; \qquad Equation 1$$
in Equation 1, C_{sulfate ion} represents the sulfate ion content in the saline soil to be tested, in wt %;
q is the mass ratio of the saline soil to be tested to the water, with no unit;
V₁ is a volume of the saline soil leachate, in mL;
V₂ is a volume of the barium chloride solution, in mL;
C₁ is a molarity of the barium chloride solution, in mol/L; and
C₂ is a molarity of the barium ions in the pretreated solution, in mol/L.

8. The method as claimed in claim 6, wherein determining a chloride ion content in the saline soil to be tested comprises
mixing the saline soil to be tested with water, and conducting solid-liquid separation to obtain the saline soil leachate, wherein a mass ratio of the saline soil to be tested to the water is denoted as q;
taking the saline soil leachate with a volume of V₃, and measuring a molarity C₃ of chloride ions in the saline soil leachate by using a chloride ion electrode-sensing analyzer; and
calculating the chloride ion content in the saline soil to be tested according to Equation 2:
$${C}_{chloride ion} = 3.55 q {C}_{3} ; \qquad Equation 2$$
in Equation 2, C_{chloride ion} represents the chloride ion content in the saline soil to be tested, in wt %;
q is the mass ratio of the saline soil to be tested to the water, with no unit; and
C₃ represents the molarity of the chloride ions measured by the chloride ion electrode-sensing analyzer, in mol/L.

9. The method as claimed in claim 6, wherein evaluating the salinization degree of the saline soil to be tested comprises
(1) under the condition that the saline soil to be tested is taken from a fine-grained soil layer,
1) under the condition that the saline soil to be tested is the chlorine saline soil or the sub-chlorine saline soil,
evaluating the saline soil to be tested as weak saline soil under the condition that the TSSC is greater than or equal to 0.3 wt % and less than 1 wt %;
evaluating the saline soil to be tested as moderate saline soil under the condition that the TSSC is greater than or equal to 1 wt % and less than 5 wt %;
evaluating the saline soil to be tested as strong saline soil under the condition that the TSSC is greater than or equal to 5 wt % and less than 8 wt %; and
evaluating the saline soil to be tested as excessive saline soil under the condition that the TSSC is greater than or equal to 8 wt %;
2) under the condition that the saline soil to be tested is the sulfite saline soil or the sulfate saline soil,
evaluating the saline soil to be tested as weak saline soil under the condition that the TSSC is greater than or equal to 0.3 wt % and less than 0.5 wt %;
evaluating the saline soil to be tested as moderate saline soil under the condition that the TSSC is greater than or equal to 0.5 wt % and less than 2 wt %;
evaluating the saline soil to be tested as strong saline soil under the condition that the TSSC is greater than or equal to 2 wt % and less than 5 wt %; and
evaluating the saline soil to be tested as excessive saline soil under the condition that the TSSC is greater than or equal to 5 wt %; and
(2) under the condition that the saline soil to be tested is taken from a coarse-grained soil layer,
1) under the condition that the saline soil to be tested is the chlorine saline soil or the sub-chlorine saline soil,
evaluating the saline soil to be tested as weak saline soil under the condition that the TSSC is greater than or equal to 2 wt % and less than 5 wt %;
evaluating the saline soil to be tested as moderate saline soil under the condition that the TSSC is greater than or equal to 5 wt % and less than 8 wt %;
evaluating the saline soil to be tested as strong saline soil under the condition that the TSSC is greater than or equal to 8 wt % and less than 10 wt %; and
evaluating the saline soil to be tested as excessive saline soil under the condition that the TSSC is greater than or equal to 10 wt %; and
2) under the condition that the saline soil to be tested is the sulfite saline soil or the sulfate saline soil,
evaluating the saline soil to be tested as weak saline soil under the condition that the TSSC is greater than or equal to 0.5 wt % and less than 1.5 wt %;
evaluating the saline soil to be tested as moderate saline soil under the condition that the TSSC is greater than or equal to 1.5 wt % and less than 3 wt %;
evaluating the saline soil to be tested as strong saline soil under the condition that the TSSC is greater than or equal to 3 wt % and less than 6 wt %; and
evaluating the saline soil to be tested as excessive saline soil under the condition that the TSSC is greater than or equal to 6 wt %.

10. The method as claimed in claim 9, wherein the fine-grained soil layer has a particle size of less than or equal to 0.075 mm, and the coarse-grained soil layer has a particle size of greater than 0.075 mm and less than or equal to 60 mm.

11. The method as claimed in claim 3, wherein the standard curve is obtained by a process comprising the steps of
providing a series of saline soil leachates with a concentration gradient having known TSSC, as standard samples to be tested; and
measuring refractive indexes of the standard samples to be tested, and plotting the standard curve with the refractive index as an abscissa and the soluble salt content as an ordinate.
